(19) United States
(12) Patent Application Publication
(10) Pub. No.: US 2025/0204985 A1
(43) Pub. Date: Jun. 26, 2025

(54) MULTI-LAYER HYDROGEL PATCH WITH ABSORBER FOR LASER TREATMENT OF A DERMATOLOGIC CONDITION

(71) Applicant: CarbonWave, Inc., Bethel, CT (US)

(72) Inventors: John B. LEE, JR., Ridgefield, CT (US); William James LEE, Ridgefield, CT (US)

(21) Appl. No.: 19/077,704

(22) Filed: Mar. 12, 2025

Related U.S. Application Data
(63) Continuation-in-part of application No. 17/946,482, filed on Sep. 16, 2022.
Application No. 17/946,482 is a continuation-in-part of application No. 17/825,234, filed on May 26, 2022.
(60) Provisional application No. 63/197,034, filed on Jun. 4, 2021.

(51) Int. Cl. A61B 18/20; A61B 18/00; A61B 18/26

(52) U.S. Cl. CPC A61B 18/203; A61B 18/26; A61B 2018/00452

(57) ABSTRACT

Some embodiments are directed to a skin treatment system that contains a laser generating device. A multi-level hydrogel patch may include a region containing an adsorbing medium (e.g., carbon black or any other substance that would have a similar effect) that, when receiving a laser beam from the laser generating device, results in Extracorporeal Shock Wave Therapy (“ESWT”) being applied to the person's skin to treat a dermatologic condition such as an epidermal or dermal tissue structure irregularity, cellulite, a stretch mark, a scar, scar-tissue, a hypertrophic scar, an acne scar, tattoo removal, conditions associated with erectile dysfunction, etc. An optic element may be provided between the laser generating device and the hydrogel patch.

### CROSS-REFERENCE TO RELATED APPLICATIONS

The present application is a continuation-in-part of U.S. patent application Ser. No. 17/946,482 entitled “SYSTEMS AND METHODS FOR LASER TREATMENT OF A DERMATOLOGIC CONDITION” and filed on Sep. 16, 2022 which was a continuation-in-part of U.S. patent application Ser. No. 17/825,234 entitled “SYSTEMS AND METHODS FOR LASER TREATMENT OF CELLULITE” and filed on May 26, 2022 which claimed the benefit of U.S. Provisional Patent Application No. 63/197,034 entitled “LASER TREATMENT OF CELLULITE” and filed on Jun. 4, 2021. The entire contents of those applications are incorporated herein by reference.

### BACKGROUND

In some cases, a herniation of subcutaneous fat within fibrous connective tissue may manifest as skin dimpling and nodularity in certain areas of a person's skin. This condition, referred to as “cellulite,” may result from hormonal factors, genetic factors, pre-disposing factors, lifestyle, etc. Many treatments have been developed for cellulite, including non-invasive therapy (mechanical suction or mechanical massage), energy-based devices (radio frequency with deep penetration of the skin, ultrasound, laser, and pulsed-light devices). More invasive subcision techniques may utilize a needle-sized microscalpel to cut through causative fibrous bands of connective tissue. Subcision procedures (manual, vacuum-assisted, or laser-assisted) are usually performed in specialist clinics. Mechanical shock wave devices may also be used to treat cellulite. All existing treatments, however, suffer from drawbacks and disadvantages.

People may similarly desire treatment for other dermatologic conditions, such as an epidermal or dermal tissue structure irregularity, a stretch mark, a scar, scar-tissue, a hypertrophic scar, an acne scar, etc. Existing treatments for such conditions also suffer from drawbacks and disadvantages.

In some cases, laser skin treatment uses adjuvant lotion, which is generally a mixture of carbon black and mineral oil. It is typically thought of as a penetrating oil that allows the carbon black to produce a thermal treatment of the target cells by penetrating and delivering the absorbent material to the target cells. We have discovered that the actual effect produced is a shock wave that disrupts the target. Moreover, carbon lotion is a messy substance. It is effective, but it gets on everything and produces smoke and vapors during treatment that require the use of a smoke evacuator. These undesirable characteristics limit the use of carbon lotion to treat skin conditions.

It would therefore be desirable to provide systems and methods that treat a dermatologic condition with a laser in a new and improved approach.

### SUMMARY

Some embodiments are directed to a skin treatment system that contains a laser generating device. A hydrogel patch may include a region, at a first side of the hydrogel patch, to be in contact with a person's skin. The region may contain an adsorbing medium that, when receiving a laser beam from the laser generating device, results in Extracorporeal Shock Wave Therapy (“ESWT”) being applied to the person's skin to treat a dermatologic condition, such as an epidermal or dermal tissue structure irregularity, cellulite, a stretch mark, a scar, scar-tissue, a hypertrophic scar, an acne scar, tattoo removal, conditions associated with erectile dysfunction, etc. An optic element may be provided between the laser generating device and the hydrogel patch.

Some embodiments comprise: means for means for placing a first side of a hydrogel patch on a person's skin, the first side including an adsorbing medium; means for generating a laser beam by a laser generating device; and means for directing the laser beam to apply ESWT to the person's skin to treat a dermatologic condition.

Some technical advantages of embodiments disclosed herein are improved systems and methods to facilitate the treatment of a dermatologic condition with a laser in a new and improved approach.

### BRIEF DESCRIPTION OF THE DRAWINGS

FIG. 1 is a high-level block diagram of a laser skin treatment system in accordance with some embodiments.

FIG. 2 is a laser skin treatment method according to some embodiments.

FIG. 3A is a laser skin treatment system in accordance with some embodiments.

FIG. 3B is a side view of a laser skin treatment system according to some embodiments.

FIG. 3C is a laser skin treatment system in accordance with another embodiment.

FIG. 3D is a side view of a laser skin treatment system according to another embodiment.

FIG. 3E is an optic element in accordance with some embodiments.

FIG. 4 illustrates acoustic wave energy created by laser reaction with a carbon patch according to some embodiments.

FIG. 5 illustrates a patch on a person's being that receives laser energy from a scanner in accordance with some embodiments.

FIGS. 6A, 6B, and 6C illustrate views of a hydrogel patch according to some embodiments.

FIGS. 7A, 7B, and 7C illustrate views of a hydrogel patch according to another embodiment.

FIGS. 8A, 8B, and 8C illustrate views of a hydrogel patch in accordance with some embodiments.

FIGS. 9A, 9B, and 9C illustrate views of a carbon patch according to some embodiments.

FIG. 10 is a laser skin treatment method according to some embodiments.

FIGS. 11A and 11B illustrate a carbon wave patch construction in accordance with some embodiments.

### DETAILED DESCRIPTION

In the following detailed description, numerous specific details are set forth in order to provide a thorough understanding of embodiments. However, it will be understood by those of ordinary skill in the art that the embodiments may be practiced without these specific details. In other instances, well-known methods, procedures, components, and circuits have not been described in detail so as not to obscure the embodiments.

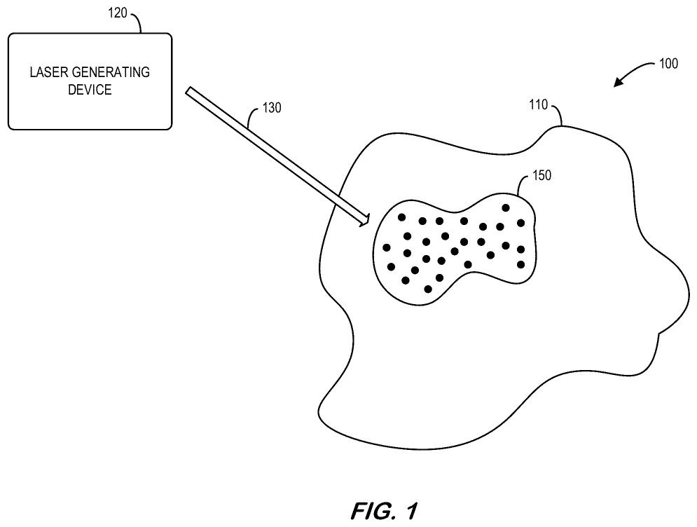

FIG. 1 is a high-level block diagram of a laser skin treatment system 100 in accordance with some embodiments. According to some embodiments, the system 100 may treat a person's skin 110 using Extracorporeal Shock Wave Therapy (“ESWT”). As used herein, ESWT may refer to a non-invasive procedure that sends powerful acoustic shock waves or pulses into soft tissue. In particular, a laser generating device 120 may direct a laser beam 130 to an adsorbing medium 150 adjacent to a person's skin 110. As used herein, the terms “adsorption” and “adsorbing” may refer to an adhesion of atoms, ions, or molecules from a gas, liquid, or dissolved solid to a surface. The process may create a film of the adsorbate on the surface of the adsorbent. Adsorption is present in many natural, physical, biological, and chemical systems and is used in many industrial applications such as heterogeneous catalysts, activated charcoal, synthetic resins, etc. In some embodiments, the adsorbing medium 150 is carbon lotion that creates an acoustic explosion strong enough to treat fascia in legs (e.g., to treat a dermatologic condition).

The adsorbing medium 150 may be a strong adsorber of the laser energy and may need to be spread on the surface of the skin 110. The laser pulse may be adsorbed quickly creating an explosive response that creates a shock wave. An optically clear material covering the adsorbing medium 150 (such that the laser beam 130 can pass through) may be used to contain the shock wave and direct it into the skin 110. The adsorbing medium 150 might be carbon black. As used herein, the phrase “carbon black” (including acetylene black, channel black, furnace black, lamp black, thermal black, etc.) may refer to a material produced by the incomplete combustion of heavy petroleum products (e.g., fluid catalytic cracking tar, coal tar, ethylene cracking tar, vegetable matter, etc.). Carbon black is a form of paracrystalline carbon that has a high surface-area-to-volume ratio (although lower than that of activated carbon). The adsorbing medium 150 could be suspended in mineral oil or in a coating material that can be applied to the person's skin 110. According to some embodiments, the treatment may be done without a hydrogel patch or with a different optically clear material that can contain the carbon black (or any other substances that would have a similar effect). A simple plastic wrap or silicone patch might be used. Note that the absorber and the laser may need to be matched. Moreover, carbon black may comprise a good absorber for many wavelengths. The laser is preferably absorbed quickly enough to create a shock wave (usually this means a plasma is formed when the absorber is irradiated by the laser). Some embodiments may use paper with black ink specifically formulated to be absorbed (e.g., ZAP-IT® laser alignment and burn paper could generate the shock wave). In some embodiments, a compressor (e.g., such as an optic element) may cause energy that would be radiated as a sound wave away from the tissue to be reflected directly back into the tissue (to magnify the shock wave effect for the carbon wave).

In this way, some embodiments may provide a carbon patch accessory device that will work with many commercially available nanosecond lasers (e.g., Q-Switched lasers typically used for tattoo removal). The patch may be affordable, easy to train, non-invasive, and effective. Some embodiments may utilize a fourth generation Q-Switched laser, such as one capable of delivering Q-Switched laser energy with an adjustable pulse rate that extends to up to 50 Pulse-Per-Second (“PPS”) and pulse energy with a super intense peak that has nanosecond level duration that can generate much shorter and more intense ESWT pulses. As used herein, the phrase “Q-Switched laser” may refer to a technique by which a laser can have extremely short pulse widths and ultra-high instantaneous peak power. Note that different pulse rates may cause different resonances in the tissue. The connective tissue's length and flexibility is a variable and selecting a pulse rate that causes a resonance that is sympathetic to the weaknesses of the connective tissue may result in a better treatment. Thus, some embodiments may utilize a laser having an adjustable PPS value.

Note that ultra short Q-Switch pulses combined with the adsorbing medium 150 may create shorter, more intense ESWT and pack more energy into a shock wave without causing cavitation. Note that mechanical means of producing ESWT may cause cavitation if too much energy is delivered. With Q-Switched laser delivery, the intensity and peak pressure can be substantially larger.

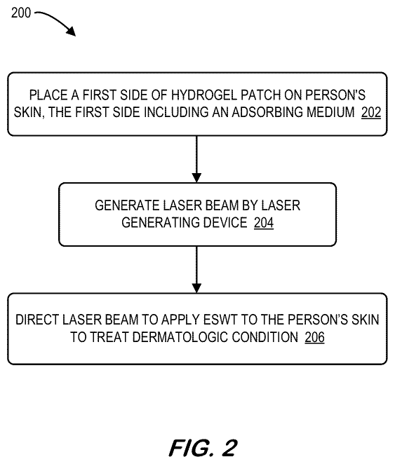

FIG. 2 is a laser skin treatment method 200 according to some embodiments. At 202, a first side of a hydrogel patch may be placed on a person's skin, the first side including an adsorbing medium. At 204, a laser beam may be generated by a laser generating device. At 206, the laser beam may be directed to apply ESWT to the person's skin to treat a dermatologic condition. The dermatologic condition might comprise, for example, an epidermal or dermal tissue structure irregularity, cellulite, a stretch mark, a scar, scar-tissue, a hypertrophic scar, an acne scar, tattoo removal, conditions associated with erectile dysfunction, etc.

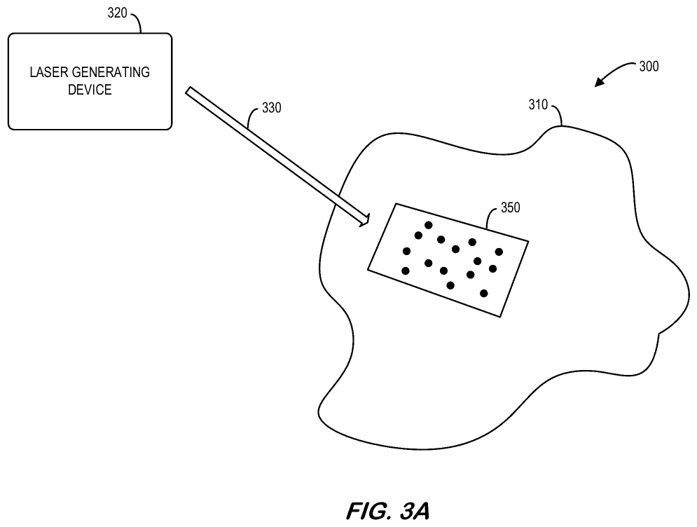

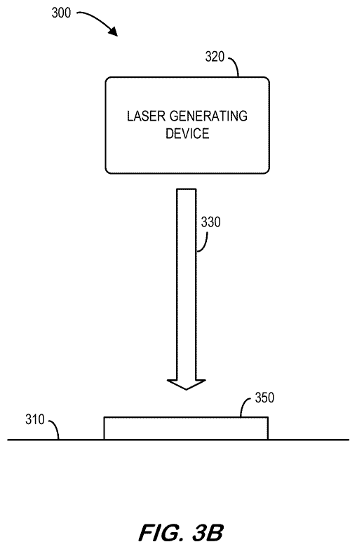

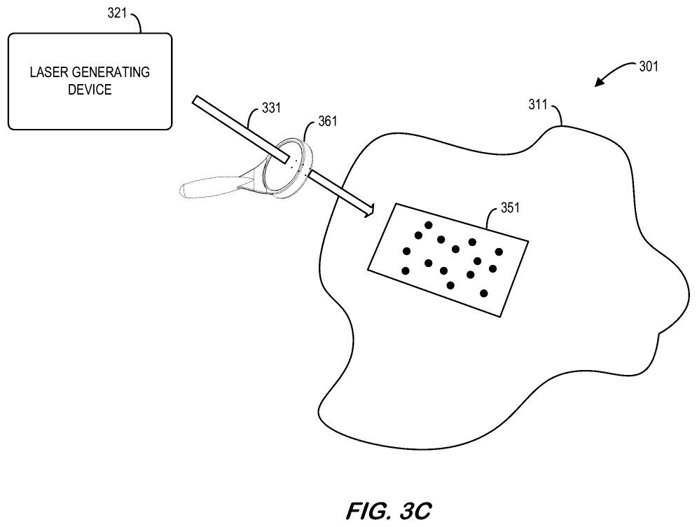

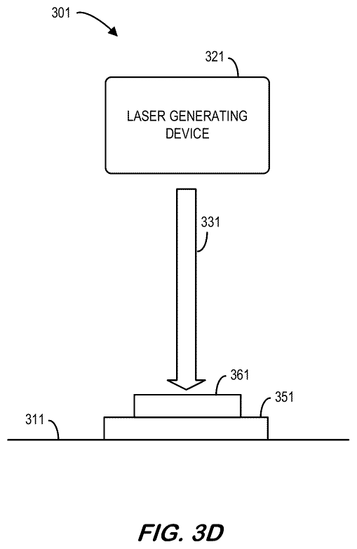

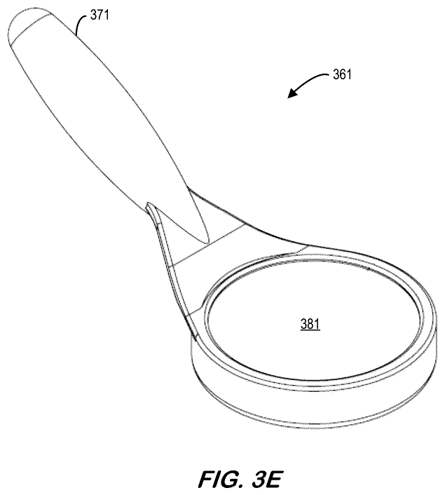

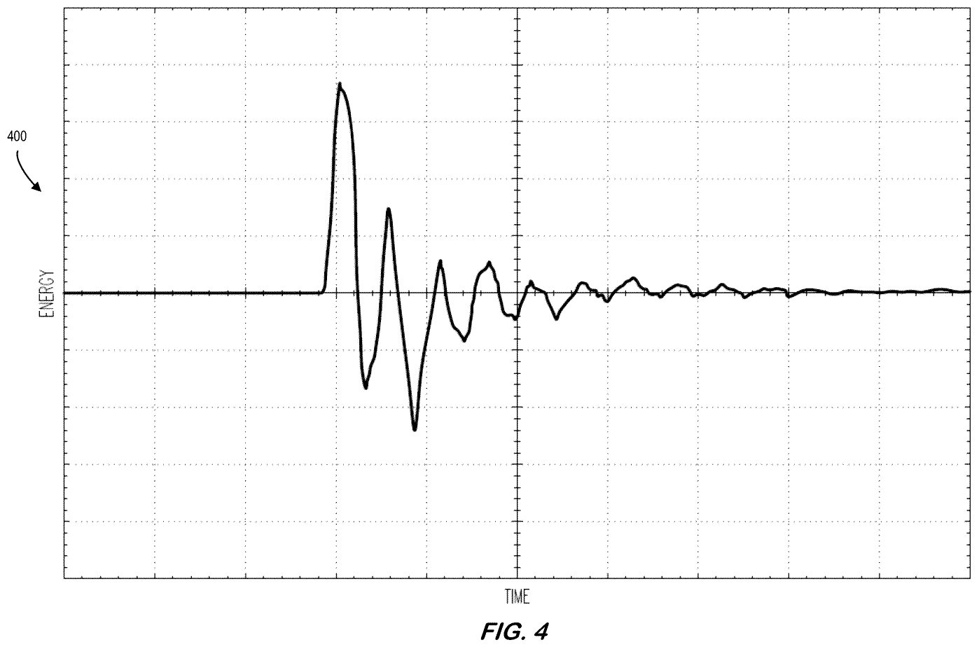

FIG. 3A is a laser skin treatment system 300 in accordance with some embodiments. As before, a laser generating device 320 directs a laser beam 330 to an adsorbing medium adjacent to the person's skin 310. In this embodiment, the adsorbing medium is a hydrogel or similar patch 350 that creates an ESWT acoustic explosion strong enough to treat a dermatologic condition. As used herein, the term “hydrogel” may refer to a material with a three-dimensional network of hydrophilic polymers that can swell in water (and hold a substantial amount of water) while maintaining the structure due to chemical or physical cross-linking of individual polymer chains. The hydrogel patch 350 can be precoated with the adsorbing medium. This makes application of the adsorber easier and enhances the treatment by helping to direct the shock wave into the skin 310. According to some embodiments, the hydrogel patch 350 is an approximately 8″ by 4″ gel sheet (with a layer of carbon black—or any other substances that would have a similar effect—between gel layers) that might be applied to a person's legs, buttocks, abdominal region, arms, etc. Different shapes and sizes of the patch 350 may be used for specific treatment areas. The laser generating device 320 results in an explosion of carbon particles that creates an acoustic sound wave that penetrates deep into tissue. For example, FIG. 4 illustrates 400 ESWT acoustic wave energy created by laser reaction with a carbon patch (with the X-axis representing time and the Y-axis representing energy). Note that encapsulating the carbon in gel sheets may help direct energy into the person's skin 310.

According to some embodiments, the laser generating device 320 utilizes a 50 Hz pulse rate. The 50 Hz pulse rate may deliver more energy as compared to typical lasers, resulting in the creation of higher pressure acoustic pulses. Note that the pulse rate might, according to some embodiments, be adjusted to generate a resonance of shock waves that corresponds to a natural resonance of target tissue. In some cases, the 50 Hz pulse rate may be too fast for a typical laser handpiece. Instead, a custom designed scanner may be used. In this case, each 8″ by 4″ hydrogel patch 350 may take approximately one to two minutes of treatment time. FIG. 5 illustrates 500 a patch 550 on a person's leg 510 receiving laser energy from a scanner 520 in accordance with some embodiments. FIG. 3B is a side view of a laser skin treatment system 300 according to some embodiments. As before, the laser generating device 320 directs the laser beam 330 to the hydrogel patch 350, with an adsorbing medium, adjacent to the person's skin 310. Note that the laser generating device 320 may run faster than other lasers (e.g., 40 or 50 pulses per second) and use a scanner. It may also have a mode that deep heats the patient's tissue to promote tightening and reduce laxity that often occurs along with cellulite.

Thus, embodiments may create an acoustic wave of energy, by using a laser interaction with carbon particles, for the treatment of a dermatologic condition. The hydrogel patch 350 is infused with carbon particles. A doctor or technician creates an acoustic wave by lasering the hydrogel patch 350. When the laser beam 330 reacts with carbon particles, it creates an acoustic wave. Acoustic waves have been studied and proven to be successful in the treatment of a dermatologic condition.

The laser generating device 320 might be associated with, according to some embodiments, lasers having a wavelength from 755 to 1064 nanometers (“nm”). The laser beam 330 may have a pulse width from 0.2 to 80 nanoseconds (“ns”) and pulse energy might range from 100 to 2500 millijoules (“mj”). The laser beam 330 may have a spot size of from 1 to 15 millimeters and a fluence of 0.1 to 5 j/cm². According to some embodiments, a larger spot size (e.g., 100 or larger) may generate a deeper penetrating shock wave. One example of a suitable laser generating device 320 might comprise a Q-Switched ND: YAG (1064 nm, pulse energy 400-2000 mj, spot size 4-12 mm, fluence 0.7 to 2.5 j/cm², and pulse width 3-12 ns). Other suitable laser generating devices 320 might be associated with wavelengths from 690 to 1200 nm, pulse widths from 0.1 ns to 1 ms, spot sizes from 1 to 15 mm, scanning areas up to 40×40 mm, fluence. 1 to 5 j/cm², and pulse energy from 100 to 4000 mj. According to some embodiments, the laser generating device 320 has an adjustable PPS rate. Such an adjustable pulse rate may allow for the tuning of ESWT pulses to target different types of tissue where the resonance is sympathetic to the weaknesses of the target tissue. In some cases, a laser beam having a PPS rate of 1 to 50 may be used for treatment. In some embodiments, a scanner might form larger constructive interference between shock waves by using a pattern that forms a wave that moves from one side of the scan area to the other. Similarly, a pattern may form a wave that moves from the edges to the center of the scan area.

FIG. 3C is a laser skin treatment system 301 in accordance with another embodiment. A laser generating device 321 directs laser beam 331 to a hydrogel patch 351, with an adsorbing medium, adjacent to a person's skin 311. In this embodiment, an optic element 361 is provided through which the beam passes. As a result of the optic element 361, energy that would be radiated as a sound wave away from the tissue is reflected directly back into the tissue. That is, the optic element 361 may act as a compressor that is used to magnify the shock wave effect for the carbon wave. Measurements of shock waves formed in tissue show that such an approach can create an intensity up to four times greater in the tissue as compared to FIGS. 3A and 3B. FIG. 3D is a side view of a laser skin treatment system according to this embodiment. As before, the laser generating device 321 directs the laser beam 331 through the optic element 361 to the hydrogel patch 351, with an adsorbing medium, adjacent to the person's skin 311. Note that the optic element 361 may be pressed against the carbon patch 351 and reflect the shock wave that is formed when the laser energy absorbed reaches the intensity to form a plasma explosion.

FIG. 3E is an optical element 361 in accordance with some embodiments. The optic element 361 may be a carbon wave compression window with a handle 371 (e.g., adapted to be held in an operator's hand angled and such that a portion of the compression window is parallel to a patient's skin) and window element 381. The window element 381 may comprise, for example, a fused silica window (e.g., with an approximately 50.8 mm diameter and 10 mm thick) bonded to the handle 371 (e.g., with Master Bond EP30MED). Note that with a Q-Switched laser, the compressor may be decoupled from the laser. This may be done, for example, by coating the window element 381 surface with an anti-reflection coating (on one or both sides of the window element 381) and/or by tilting it relative to the laser beam. In some embodiments, such a feature is built into a laser's scanning hand piece. The window element 381 may be thick enough to withstand the shock wave forces and may compress the skin and reflect the shock waves that would normally propagate away from the skin back into the target tissue. Note that coatings can be used to reduce optical losses entering and leaving a compressor. With some lasers, a coating on the output side of a compressor might be damaged by the laser beam and patch interaction. Therefore, a coating may only be needed on the input side of a compressor for that type of laser beam.

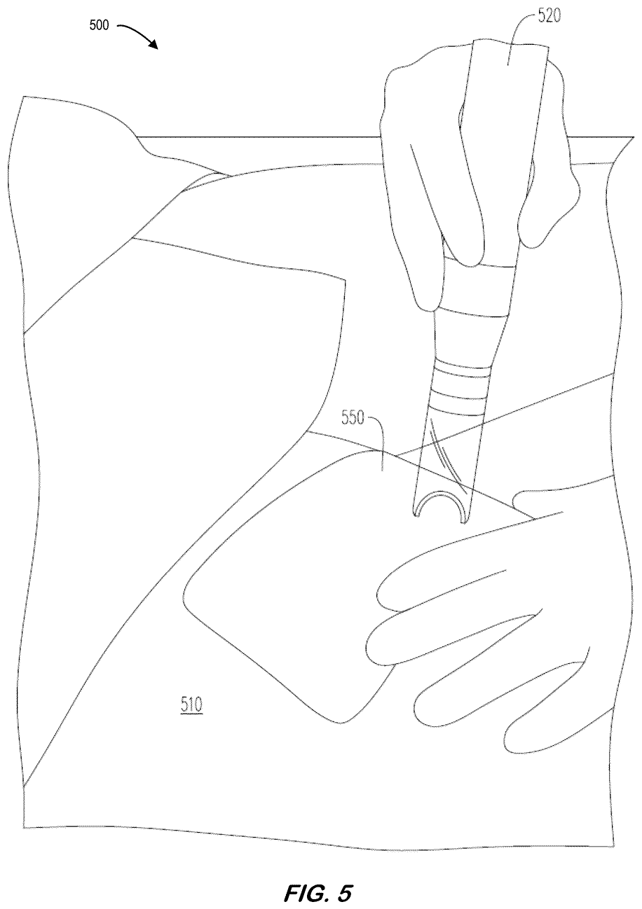

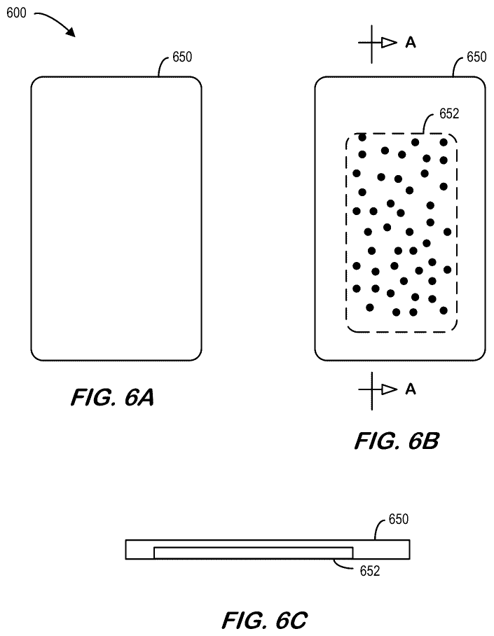

FIGS. 6A, 6B, and 6C illustrate views of a hydrogel patch 650 according to some embodiments. In particular, FIG. 6A is view 600 of the top of the hydrogel patch 650 and FIG. 6B is a view of the bottom of the hydrogel patch 650 (the side applied to a person's skin). As can be seen, a portion 652 of the bottom side has been infused with carbon particles. FIG. 6C is a cross-sectional side view of the hydrogel patch 650 taken along cross section line AA of FIG. B. The carbon particle infused portion 652 of the patch 650 would therefore be placed directly up against a person's skin in an area to be treated for a dermatologic condition.

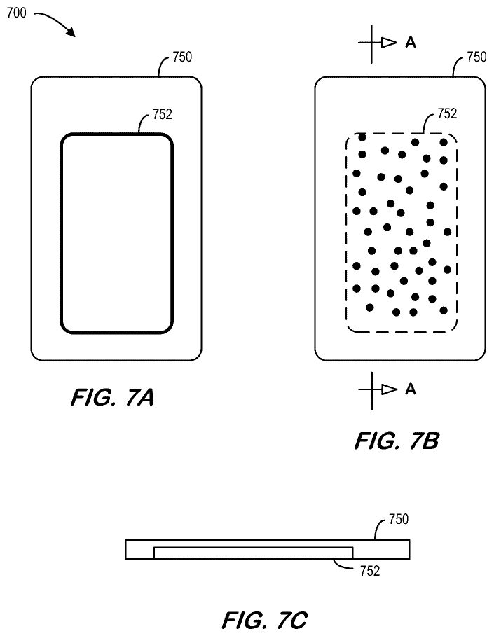

FIGS. 7A, 7B, and 7C illustrate views of a hydrogel patch 750 according to another embodiment. As before, FIG. 7A is view 700 of the top of the hydrogel patch 750 and FIG. 7B is a view of the bottom of the hydrogel patch 750 (the side applied to a person's skin). As can be seen, a portion 752 of the bottom side has been infused with carbon particles. FIG. 7C is a cross-sectional side view of the hydrogel patch 750 taken along cross section line AA of FIG. B. The carbon particle infused portion 752 of the patch 750 would therefore be placed directly up against a person's skin in an area to be treated for a dermatologic condition. Note that the entire surface of the hydrogel patch 750 does not contain the infused portion 752. As a result, markings on the top of the hydrogel patch 750 may help guide a doctor or other professional to exactly where the laser beam should be focused.

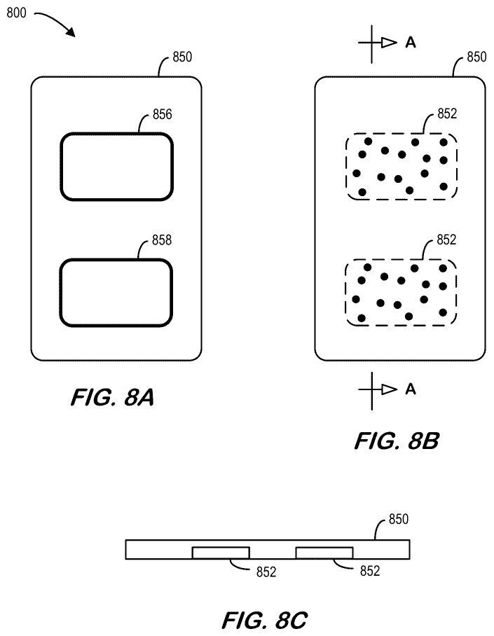

Similarly, FIGS. 8A, 8B, and 8C illustrate views of a hydrogel patch 850 in accordance with some embodiments. Again, FIG. 8A is view 800 of the top of the hydrogel patch 850 and FIG. 8B is a view of the bottom of the hydrogel patch 850 (the side applied to a person's skin). As can be seen, two separate portions 852 of the bottom side have been infused with carbon particles. FIG. 8C is a cross-sectional side view of the hydrogel patch 850 taken along cross section line AA of FIG. 8B. The two carbon particle infused portions 852 of the patch 850 would therefore be placed directly up against a person's skin in an area to be treated for cellulite. Note that once again, the entire surface of the hydrogel patch 850 does not contain the two infused portions 852. As a result, multiple markings 856, 858 on the top of the hydrogel patch 850 may help guide a doctor or other professional to where the laser beam should be focused.

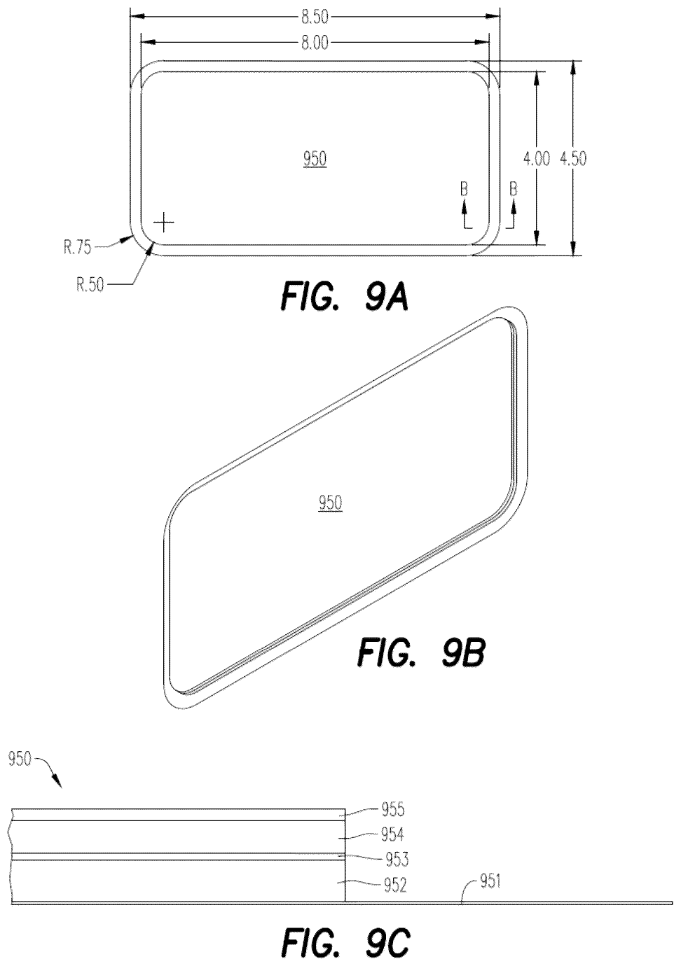

FIGS. 9A, 9B, and 9C illustrate views of a carbon patch 950 according to some embodiments (distances in inches). In particular, FIG. 9A is a view of the top of the carbon patch 950 and FIG. 9B is a perspective view of the carbon patch 950. FIG. 9C is a side view illustrating layers of the carbon patch 950. A first layer 951 may represent a release layer and may comprise a clear evape film material. A second layer 952 may represent a hydrogel material layer. A third layer 953 may represent a carbon layer and a fourth layer 954 may represent another hydrogel material layer (e.g., similar to the second layer 952). According to some embodiments, the second layer 952 may be thinner than the fourth layer 954 to improve performance. In still other embodiments, the fourth layer 954 may be eliminated entirely (that is, the fourth layer 954 may be optional). A fifth layer 955 may represent a cover layer. According to some embodiments, carbon patches 950 may be designed and/or shaped for treatment of specific tissue areas, such as a patient's cheek or neck area.

In some embodiments, a hydrogel patch may have carbon black distributed through the gel. In other embodiments, a discrete layer of highly concentrated carbon black may be provided close to the skin. Being highly concentrated (as opposed to diluted throughout the hydrogel), may result in a more concentrated plasma and a bigger, more intense shock pulse. For example, a carbon black layer may be less than 0.005″ thick. Note that an absorber might be a layer of carbon black on a sub-straight other than hydrogel (and may be a compressed absorber that is contained during treatment by a compressor. In some embodiments, a compressor may be coated by an adsorbing ink, or a patch may comprise a clear plastic with ink coated on one side. Some embodiments may comprise a compressor with a clear material having an absorbing coating on one side. Other embodiments may include a patch constructed of a material other than hydrogel with a coating or layer of absorber. A patch may, or may not, include an adhesive to hold it in place. Other embodiments involve an optic with a liquid absorber that is actively injected in between a patient's skin and a compressor.

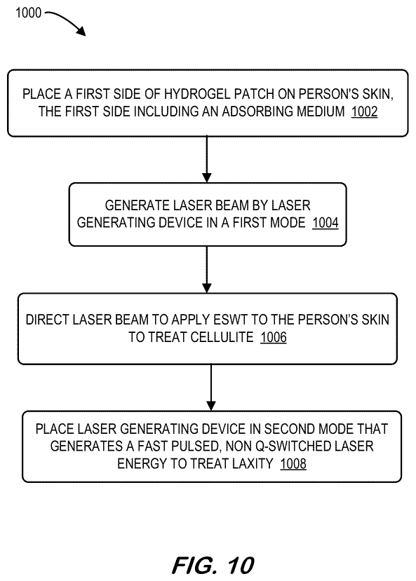

Note that skin laxity is a common problem in areas where cellulite is formed. As used herein, the phrase “skin laxity” might be associated with, for example, a loss of skin elasticity, a loosening of connective tissue framework, and/or a deepening of skin folds resulting in prominence of submandibular and submental tissues. To address such problems, FIG. 10 is a laser skin treatment method 1000 according to some embodiments. At 1002, a first side of a hydrogel patch may be on a person's skin, the first side including an adsorbing medium. At 1004, a laser beam may be generated by a laser generating device operating in a first mode. At 1006, the laser beam may be directed to apply ESWT to the person's skin to treat cellulite. At 1008, the laser generating device is in a second mode that generates a fast pulsed, non Q-Switched laser energy that can treat laxity by gently heating the area. This may be substantially more effective when the laser pulse rate is higher and the laser power is absorbed non-specifically in the tissue. In some embodiments, the laser device enters the second mode immediately after the carbon patch is used (e.g., by either automatically or manually selecting the second mode). Such an embodiment may better satisfy the needs of cellulite skin.

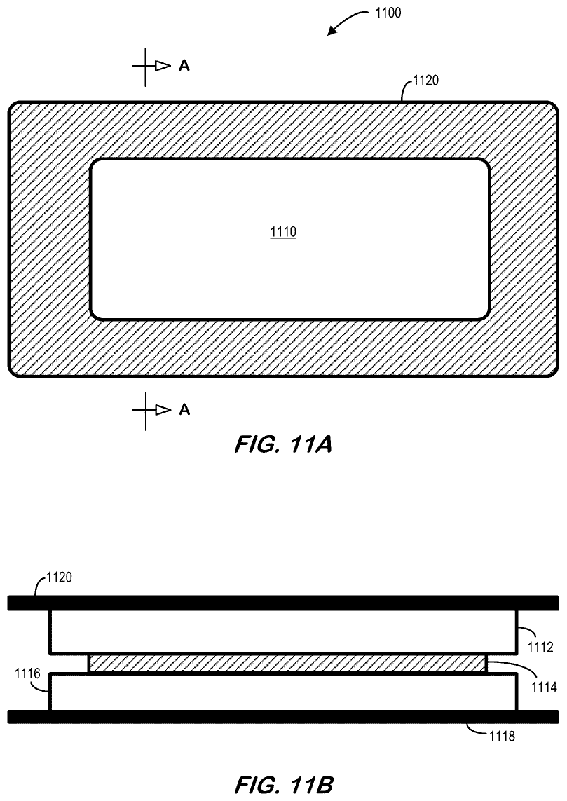

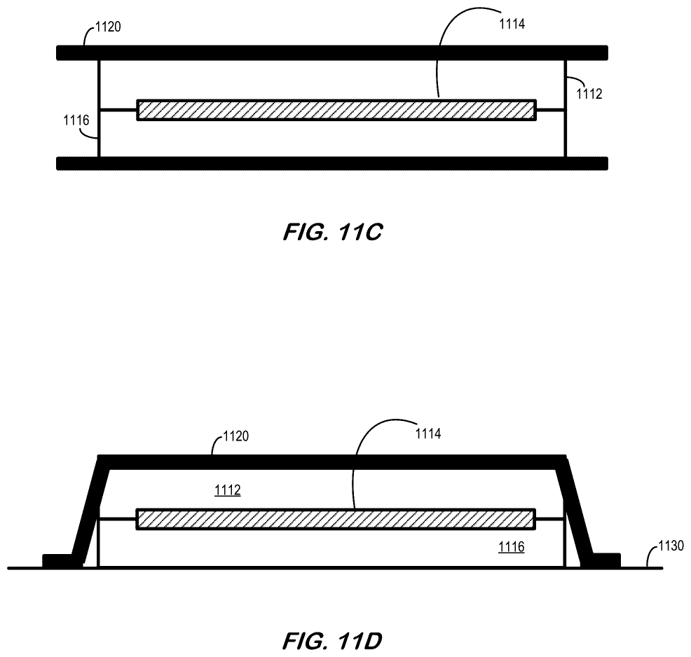

FIGS. 11A and 11B illustrate a carbon wave patch construction in accordance with some embodiments. FIG. 11A is view 1100 of the top of an active area 1110 of an entire carbon wave patch 1120, and FIG. 11B is a cross-sectional side view of the carbon wave patch 1120 taken along cross section line AA of FIG. A. The area between the active area 1110 and the entire wave patch 1120 (cross-hatched in FIG. 11A) may comprise a border area that helps contain smoke and vapors.

At the very top (opposite a person's skin), a protective layer may be provided covering the entire carbon wave patch 1120 as shown in FIG. 11B. Beneath that, a layer of clear hydrogel 1112 is provided followed by a thin film layer coated with absorber 1114 (which may be fixed in wax). Another layer of clear hydrogel 1116 is provided followed by a removable film liner 1118 that is taken off the patch immediately before the patch is applied to a person's skin. FIG. 11C illustrates an entire carbon wave patch 1120 in which the first and second hydrogel layers 1112, 1116 are compressed and/or expanded to completely enclose the thin film layer coated with absorber 1114. FIG. 11D illustrates an entire carbon wave patch 1120 with the removable film liner 1118 taken off, and the patch 1120 has been applied to the person's skin 1130. As in FIG. 11C, the first and second hydrogel layers 1112, 1116 are compressed and/or expanded to completely enclose the thin film layer coated with absorber 1114 further reducing production of smoke and vapors.

By embedding the absorber in a patch that lets laser light pass into it, the mess of carbon lotion may be eliminated. However, the shock wave produced may be weak because the concentration of the absorber generally distributed through hydrogel produces a distributed response over the thickness of the material resulting in a pulse with a distributed pressure profile. Essentially, the profile is a long wavelength with a peak intensity that is much lower but distributed over a larger volume than the pressure wave produced by a carbon lotion spread over the skin.

The density of the absorber, the strength of the absorption coefficient (absorber/wavelength), the pulse width, and the intensity of the light may impact the profile. When constructing a patch, the absorbing medium should be in a highly concentrated layer. Embedding the absorber in a patch also manages smoke and vapors. In addition, a border of at least 0.2″ (e.g., preferably 0.3″ or more) with the hydrogel extending beyond the area under the patch where the absorber is. This arrangement lets the adhesion of the patch seal the edges, keeping the smoke and vapors under the patch.

To increase the density of the absorption layer, two separate layers of hydrogel 1112, 1116 surround the thin layer with the absorber 1114. To accomplish this in a volume manufacturing process, the absorber may be a dry coating on a thin flexible sheet material that can roll feed into the manufacturing process. A very thin plastic sheet with a layer of absorber coated onto one surface may be used. For example, a carbon black wax coating on a thin plastic sheet may be employed.

The depth of penetration of the shock wave produced by the laser action on the patch is related to the size of the laser spot. In some embodiments, a spot greater than 5 mm in diameter creates a wave front in the central section of the spot. It continues straight until an edge effect starts to dissipate the pulse laterally. If the spot is less than 5 mm in diameter, edge effects may reduce penetration and lower efficacy. According to some embodiments, the spot size is 6 mm or greater.

The intensity of the beam may also be important—at least 2 J/cm² may be provided for the entire spot area to generate an intense wave. The energy may also be delivered in a short pulse (e.g., less than 8 nanoseconds). The short pulse may enhance the wave and create a sharp leading edge to the pulse that may be more effective treating skin conditions.

A scanner-controlled pulse pattern with a pulse rate greater than 20 pulses per second may be utilized to produce constructive interference within the tissue increasing stress on tissue to disrupt the cellulite causing septa. The scanner can direct the pulses in patterns that reinforce by moving from one side to the opposite side or from the edges to the center.

Typically, the concept of embedding an absorber in a patch is conceived as dissolving the absorber uniformly throughout the patch or throughout a portion of the volume of the patch. Some embodiments described herein have a concentrated layer in the patch where the absorber is compressed into a small volume. In the typical case, the light from the laser beam is intense enough to illuminate and be absorbed by the full volume of the patch. The energy in the laser beam is a constant and the absorption action converts the light energy into a plasma explosion that is distributed throughout the volume where the absorber is distributed.

The explosive energy is distributed over this volume, and the thickness of the absorber layer defines the wavelength of the shock wave, and the amplitude of the wave is defined by the local concentration of the absorber. It may be important to concentrate the absorber into a dense thin layer. In this way, the amplitude of the response may be improved, and the shock wave pulse width may be shortened to be equal to the thickness of the absorbing layer.

According to some embodiments, carbon black is deposited in a layer that is a few microns of nearly pure absorber and is dense enough to absorb the energy in a very short, very intense laser pulse. Using a Q-switched Nd:YAG laser with a pulse width of less than 6 nanoseconds and a per pulse energy of at least 500 millijoule and a spot fluence of 2 joules per square centimeter will deposit a plasma energy of 83 megajoules, and shorter pulse, than other shock wave generators as well as a much higher amplitude to improve clinical results.

Spot size may also be important. A flat uniform layer of absorber activated by the laser energy becomes a wave front that propagates directly into the tissue. If the spot is very large, the wave front continues directly. With a small spot, the wave front dissipates at the edges and the energy propagates into the tissue and away from the spot (dissipating the wavefront intensity).

With respect to shock wave pulse width, the transverse and longitudinal shape of the shock wave is governed by the geometry of the patch construction. When intense laser light impinges on an absorber and the intensity is high enough to form a plasma, the plasma forms in the shape of the absorber. In this case, it is a thin layer that covers the full laser spot size. To form an intense shock wave, the laser light must illuminate the absorber. In the case of a patch, the full thickness of the carbon layer is illuminated at the speed of light. When the full thickness is converted to a plasma (e.g., within a few nanoseconds) a shock wave is formed with the initial shape of the illuminated layer of absorber. The speed of propagation of a sound or shock wave has been measured to be between 1525 and 1590 m/s. The wave front created by the laser light plasma conversion propagates through the tissue at that speed and the wavelength of the shock wave front perpendicular to the spot is equal to the thickness of the absorber layer. If the absorber layer is 0.1 mm thick, then the wave front has a rise time of 65 nanoseconds. If the absorber layer is 0.2c mm thick, the rise time is 130 nanoseconds. Because both wave fronts originate from the same laser beam absorber interaction, they contain an equal amount of energy—but the thinner layer allows the wave front energy to be delivered in a shorter period of time. Thus, the thinner the absorber layer is, the more compressed the wave front and the larger the peak pressure is applied (e.g., half the thickness delivers twice the peak pressure). This is a method to increase the effectiveness of the shock wave.

Thus, embodiments may provide full body rejuvenation. Moreover, some embodiments may target superficial and deeper skin structures. The present invention has been described in terms of several embodiments solely for the purpose of illustration. Persons skilled in the art will recognize from this description that the invention is not limited to the embodiments described but may be practiced with modifications and alterations limited only by the spirit and scope of the appended claims.

1. A skin treatment system, comprising:
a laser generating device; and
a hydrogel patch, including:
a protective layer,
a first layer of clear hydrogel adjacent to and beneath the protective layer,
a thin film layer coated with absorber adjacent to and beneath the first layer of clear hydrogel,
a second layer of clear hydrogel adjacent to and beneath the thin film layer, and
a removable film liner adjacent to and beneath the second layer of clear hydrogel, wherein the removable film layer is to be taken off the patch immediately before the patch is applied to the person's skin,
wherein the hydrogel patch, when receiving a laser beam from the laser generating device, results in Extracorporeal Shock Wave Therapy (“ESWT”) being applied to a person's skin to treat a dermatologic condition.

2. The system of claim 1, wherein the dermatologic condition comprises at least one of: (i) an epidermal or dermal tissue structure irregularity, (ii) cellulite, (iii) a stretch mark, (iv) a scar, (v) scar-tissue, (vi) a hypertrophic scar, (vii) an acne scar, (viii) tattoo removal, and (ix) a condition associated with erectile dysfunction.

3. The system of claim 1, wherein the laser beam has a pulse width from 0.2 to 80 nanoseconds (“ns”), a pulse energy from 100 to 2500 millijoules (“mj”), a spot size of from 6 to 15 millimeters, and a fluence of 2 to 5 j/cm².

4. The system of claim 1, wherein the laser beam has an adjustable Pulse-Per-Second (“PPS”) rate of 20 to 50.

5. The system of claim 3, wherein the laser beam delivers energy in pulses less than 8 nanoseconds in duration.

6. The system of claim 5, wherein the laser beam directs pulses in a reinforcing pattern from one side of a spot to an opposite side of the spot.

7. The system of claim 5, wherein the laser beam directs pulses in a reinforcing pattern from an edge of a spot to center of the spot.

8. The system of claim 1, wherein the hydrogel patch contains carbon black or any other substance that would have a similar effect.

9. The system of claim 8, wherein the hydrogel patch is approximately 8 inches by 4 inches.

10. The system of claim 8, wherein the laser generating device includes a scanner to provide 50 Hz Q-Switched laser energy to the person's skin.

11. The system of claim 10, wherein the laser generating device generates the Q-Switched laser energy in a first mode and further has a second mode that generates a fast pulsed, non Q-Switched laser energy that treats laxity by gently heating the person's skin.

12. A method to treat a dermatologic condition, comprising:
placing a hydrogel patch on a person's skin, the hydrogel patch including:
a protective layer,
a first layer of clear hydrogel adjacent to and beneath the protective layer,
a thin film layer coated with absorber adjacent to and beneath the first layer of clear hydrogel,
a second layer of clear hydrogel adjacent to and beneath the thin film layer, and
a removable film liner adjacent to and beneath the second layer of clear hydrogel, wherein the removable film layer is to be taken off the patch immediately before the patch is applied to the person's skin;
generating a laser beam by a laser generating device; and
directing the laser beam, through the hydrogel patch, to apply Extracorporeal Shock Wave Therapy (“ESWT”) to the person's skin to treat a dermatologic condition.

13. The method of claim 12, wherein the dermatologic condition comprises at least one of: (i) an epidermal or dermal tissue structure irregularity, (ii) cellulite, (iii) a stretch mark, (iv) a scar, (v) scar-tissue, (vi) a hypertrophic scar, (vii) an acne scar, (viii) tattoo removal, and (ix) a condition associated with erectile dysfunction.

14. The method of claim 12, wherein the laser beam has a pulse width from 0.2 to 80 nanoseconds (“ns”), a pulse energy from 100 to 2500 millijoules (“mj”), a spot size of from 6 to 15 millimeters, and a fluence of 2 to 5 j/cm².

15. The method of claim 12, wherein the laser beam has an adjustable Pulse-Per-Second (“PPS”) rate of 20 to 50.

16. The method of claim 12, wherein the hydrogel patch contains carbon black or any other substance that would have a similar effect.

17. The method of claim 16, wherein the laser generating device includes a scanner to provide 50 Hz Q-Switched laser energy to the person's skin.

18. The method of claim 17, wherein the laser generating device generates the Q-Switched laser energy in a first mode and further has a second mode that generates a fast pulsed, non Q-Switched laser energy that treats laxity by gently heating the person's skin.

19. The method of claim 12, wherein the laser beam delivers energy in pulses less than 8 nanoseconds in duration.

20. The method of claim 19, wherein the laser beam directs pulses in a reinforcing pattern from one side of a spot to an opposite side of the spot.

21. The method of claim 19, wherein the laser beam directs pulses in a reinforcing pattern from an edge of a spot to center of the spot.

22. A patch to be applied to a person's skin, comprising:
a protective layer;
a first layer of clear hydrogel adjacent to and beneath the protective layer,
a thin film layer coated with absorber adjacent to and beneath the first layer of clear hydrogel;
a second layer of clear hydrogel adjacent to and beneath the thin film layer; and
a removable film liner adjacent to and beneath the second layer of clear hydrogel, wherein the removable film layer is to be taken off the patch immediately before the patch is applied to the person's skin.

23. The patch of claim 22, wherein the thin film layer is coated on one surface with dry carbon black.
